(19) United States
(12) Patent Application Publication
(10) Pub. No.: US 2008/0221567 A1
(43) Pub. Date: Sep. 11, 2008

(54) ELECTROSURGICAL TISSUE REMOVAL WITH A SELECTIVELY INSULATED ELECTRODE

(75) Inventors: Robert Sixto, Miami, FL (US); Charles D. Lennox, Hudson, NH (US)

Correspondence Address: COOLEY GODWARD KRONISH LLP;ATTN: Patent Group, Suite 1100, 777 - 6th Street, NW, WASHINGTON, DC, 20001, US

(73) Assignee: Boston Scientific Scimed, Inc., Maple Grove, MN (US)

(21) Appl. No.: 12/027,859

(22) Filed: Feb. 7, 2008

Related U.S. Application Data
(62) Division of application No. 10/292,162, filed on Nov. 12, 2002, now abandoned.
Application No. 10/292,162 is a division of application No. 09/359,335, filed on Jul. 21, 1999, now abandoned.
Application No. 09/359,335 is a continuation-in-part of application No. 08/940,665, filed on Sep. 30, 1997, now Pat. No. 6,494,881.

(51) Int. Cl. A61B 18/14

(52) U.S. Cl. 606/41

(57) ABSTRACT

An electrosurgical device includes an elongated body including a proximal end and a distal end and defining a longitudinal axis, at least one arm coupled to the distal end of the elongated body and an electrode coupled to at least one arm. The electrode includes an upper surface and a lower surface. The lower surface is substantially convex and defines a radius of curvature relative to an axis substantially perpendicular to the longitudinal axis.

### CROSS-REFERENCE TO RELATED APPLICATION

This is a continuation-in-part of U.S. patent application Ser. No. 08/940,665 filed Sep. 30, 1997, the entirety of which is incorporated herein by reference.

### TECHNICAL FIELD

This invention relates to electrosurgical devices, and more particularly to improved electrosurgical devices having selectively insulated portions for use in procedures such as resection, incision, ablation, and/or coagulation.

### BACKGROUND INFORMATION

There are many medical procedures in which tissue is removed for diagnostic or therapeutic reasons. For example, transurethral resection of the prostate (TURP) is performed to treat benign or cancerous prostatic hyperplasia, which blocks the urethra. Transurethral resection may also be performed in the bladder (TURB). In a transurethral resection procedure, a resection electrode is inserted into the urethra of a patient through a resectoscope. An electric current applied through the electrode heats the prostate tissue sufficiently to break inter-cellular bonds, thereby cutting or resecting the tissue. Extensive bleeding can occur as a result of resection, and the bleeding can obstruct the physician's view and/or lead to dangerous blood loss levels. Coagulation of the resected tissue can minimize bleeding.

Prior to and during the resection and coagulation procedures, a fluid inserted through the resectoscope irrigates the treatment region. Irrigation displaces urine in the urethra and distends the urethra to create a working space. During the resection procedure, irrigation displaces removed tissue and blood. Examples of suitable irrigation fluids include distilled water (i.e., deionized water), glycine, sorbitol, and saline. An advantage of using saline over the other irrigation fluids is that saline prevents side effects known as TURP syndrome. TURP syndrome occurs in 2-3% of patients undergoing prostate resection. TURP syndrome is caused by rapid absorption of electrolyte free fluid, which may lead to mental confusion, nausea, visual disturbance, cardiac arrhthmias, or central nervous system dysfunction. Complication from TUR syndrome can lead to death. A disadvantage of using saline, however, is that the electrolytic nature of the saline results in an increased conductivity through the irrigant. It is often difficult to generate a plasma field, necessary to resect tissue, at the tip of an electrode because current applied to the electrode quickly diffuses toward the saline, instead of traveling directly into the tissue. Moreover, an RF generator in communication with the electrode will sense that a short circuit is present at the electrode tip, because saline provides a low initial impedance across the output leads. Therefore, the output voltage starts low and then builds up as the RF generator learns that an impedance exists at the tip. The impedance builds up as the electrode is heated, causing the fluid in contact with the electrode to vaporize. The result is then an increase in the impedance of the system. The RF generator responds by increasing the amount of power delivered. This continues in the manufacturer's specified working impedance range. Above this range, the RF generator delivers decreasing amounts of power.

Existing electrosurgical devices tend to be inefficient when used with an electrolytic fluid such as saline. As a result, resection performed with existing devices is either inadequately carried out, or a greater amount of energy must be applied to the electrode to perform resection, which raises other concerns. Adjacent healthy tissues may be damaged during the resection procedure when a large amount of energy is applied.

### SUMMARY OF THE INVENTION

An object of the invention is to provide an electrosurgical device which overcomes these problems by being able to focus energy emission towards the tissue, reducing energy loss to the resected chips or the fluid delivered to the tissue site, while avoiding the need for higher power levels to achieve such an effect. Another object of the invention is to provide an electrosurgical device which provides an increase in current density at the electrode, and an electrode that is capable of generating plasma fields in a tissue being irrigated with fluid, such as, for example, a non-osmotic fluid (e.g., saline, glycine, sorbitol), without being embedded within tissue. Lower power levels can be used with the electrosurgical devices of the present invention in performing resection procedures, since diffusion of energy at the distal tip of the resecting electrode has been reduced.

The present invention features an electrosurgical device comprising an elongated body including a proximal end and distal end and defining a longitudinal axis, at least one arm coupled to the distal end of the elongated body, and an electrode coupled to at least one arm. The electrode includes an upper surface and a lower surface. The lower surface is substantially convex and defines a radius of curvature relative to an axis substantially perpendicular to the longitudinal axis.

In one embodiment the upper surface of the electrode is smaller than the lower surface of the electrode. In another embodiment the upper surface of the electrode is substantially concave. Alternatively, the upper surface of the electrode can be substantially flat.

In another embodiment, the upper surface of the electrode includes an insulative coating. An example of an insulative coating is a ceramic coating. Ceramic coatings can comprise materials such as alumina, zirconia, and combinations such as alumina and titanic The preferable thickness of the ceramic coating is from about 0.0002 inches to about 0.03 inches. In still another embodiment, the lower surface of the electrode comprises a ceramic base material and a metallic coating disposed over the ceramic base material.

In another aspect, the invention comprises a method of manufacturing an electrosurgical device. According to the method, an electrode coupled to an elongated body is provided and a ceramic coating is sprayed over an upper surface of the electrode. The electrode comprises a conductive member. The electrode includes an upper surface and a lower surface. The lower surface is substantially convex with a radius of curvature relative to an axis substantially perpendicular to a longitudinal axis of the elongated body. In one embodiment, a bond coating is placed on the conductive member prior to spraying. The preferable thickness of the ceramic coating is from about 0.0002 inches to about 0.03 inches.

In another embodiment, the ceramic coating is applied by thermal spraying. Thermal spraying can also be performed using a high velocity fuel spraying method. In yet another embodiment, the upper surface of the electrode is roughened prior to thermal spraying. One method of roughening the upper surface is by sand blasting it. In still another embodiment, thermal spraying of the upper surface can also be accomplished with alumina coating or alumina and titania coating. In a further embodiment, the ceramic coating is applied by plasma spraying.

The foregoing and other objects, features, and advantages of the invention will become apparent from the following, more particular description of the preferred embodiments of the invention.

### BRIEF DESCRIPTION OF THE DRAWINGS

This invention is described with particularity in the appended claims. The above and further advantages of this invention may be better understood by referring to the following description taken in conjunction with the accompanying drawings.

FIG. 1a is a perspective view of an electrosurgical device having a broad loop electrode.

FIG. 1b is an enlarged perspective view of a distal portion of the electrosurgical device of FIG. 1a.

FIG. 2 is a perspective view of another electrosurgical device having a broad loop electrode.

FIG. 3 is a cross section view of another electrosurgical device having a broad loop electrode.

FIG. 4a is a side view of an electrosurgical device having a standard loop electrode.

FIG. 4b is a perspective view of an electrosurgical device having a horizontal loop electrode.

FIG. 5a is a perspective view of an electrosurgical device having a crescent shape electrode.

FIG. 5b is a side view of another electrosurgical device having a crescent shape electrode.

FIG. 5c is a side view of another electrosurgical device having a crescent shape electrode.

FIG. 6 is a side view of an electrosurgical device having a knife electrode.

FIG. 7a is a side view of an electrosurgical device having a grooved roller electrode.

FIG. 7b is a side view of another electrosurgical device having a grooved roller electrode.

FIG. 8a is a perspective view of another electrosurgical device having a broad loop electrode.

FIG. 8b is an enlarged perspective view of a distal portion of the electrosurgical device of FIG. 8a.

FIG. 9a is a perspective view of an electrosurgical device having a cylindrical roller electrode.

FIG. 9b is a perspective view of an electrosurgical device having a spherical roller electrode.

FIG. 10a is a perspective view of another electrosurgical device having a broad loop electrode.

FIG. 10b is an enlarged perspective view from a proximal side of a distal portion of the electrosurgical device of FIG. 5a.

FIG. 11a is a cross-sectional view of a dual ion beam deposition chamber for depositing an insulative coating on an electrode.

FIG. 11b is a cross-sectional view of a high velocity oxygen fuel thermal spray chamber.

FIG. 11c is a cross-sectional view of a plasma thermal spray chamber.

FIG. 12 is a side view illustrating selective resection and cauterization of prostate tissue using the electrosurgical device of the present invention.

### DETAILED DESCRIPTION

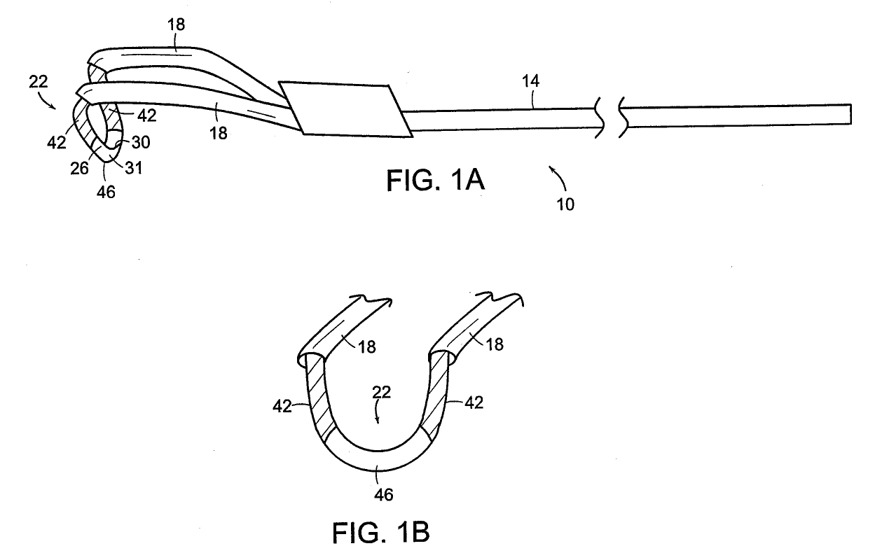

Referring to FIGS. 1a and 1b, a device 10 includes an elongated body 14, a pair of arms 18 extending from a distal end of the elongated body 14, and a broad loop electrode 22 connecting the pair of arms 18. U.S. Pat. No. 5,569,244 incorporated herein by reference describes the structure of the broad loop electrode 22. The broad loop electrode 22 is capable of both resecting and coagulating tissue. The pair of arms 18 comprises an electrical lead and an insulative sheath contains the leads. The proximal end of the elongated body 14 is adapted to be coupled to an energy source (not shown). Suitable conductive materials for forming the broad loop electrode 22, include, for example, stainless steel, tungsten, titanium, aluminum, brass, silver alloy, copper alloy, as well as other materials exhibiting conductive properties. The broad loop electrode 22 defines a pair of end sections 42 and a base section 46. Each end section 42 is coupled to an arm 18 and can comprise the conductive material having an insulative coating or sheath disposed thereon as further described. The base section 46 lies between the end sections 42 and, in the present embodiment, comprises the conductive material without the insulative coating. The base section 46 is the first region to be contacting the target tissue during a procedure. In this embodiment, energy applied to the electrode 22 remains focused at the base section 46 when the device 10 is used along with an electrolytic fluid such as, for example, saline.

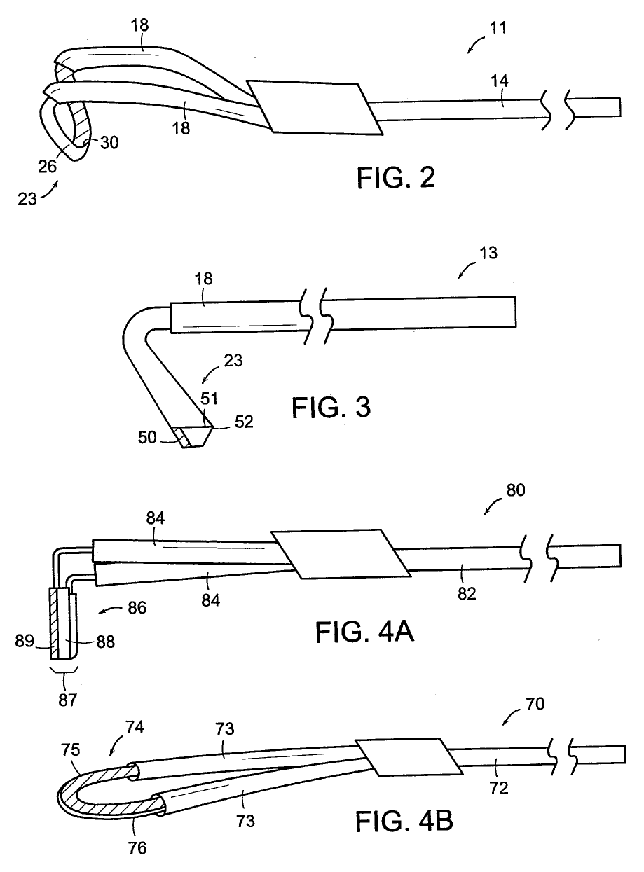

Referring to FIG. 2, an electrosurgical device 11 includes an elongated body 14, a pair of arms 18 in communication with a distal end of the elongated body 14, and a broad loop electrode 23 in communication with the pair of arms 18. The elongated body 14 includes an electrode lead (not shown) for transporting an electrical energy from a power source (not shown) to the broad loop electrode 23. The broad loop electrode 23 has an upper surface 30 covered with an insulative coating and a lower surface 26 without the insulative coating. The lower surface 26 exposes the conductive material with which the electrode 23 is constructed. In this embodiment, energy remains focused at the lower surface 26, which comes in contact with tissue during coagulation and/or resection. The conductive material forming the upper surface 30 of the electrode 23 which does not contribute to resection or coagulation, is covered with the insulative coating to prevent energy dissipation through the upper surface 30.

Referring to FIG. 3, an electrosurgical device 13 includes a broad-loop electrode 23. The broad loop electrode 23 has a distal edge 50 and a proximal edge 52. The distal edge 50 is covered with the insulative coating and the proximal edge 52 is uncovered, exposing the conductive material forming the electrode 23. In this embodiment, the proximal edge 52 focuses energy for resecting tissue. In another embodiment, both the distal edge 50 and an inner surface 51 of the electrode 23 are covered with the insulative coating to reduce the exposed conductive surface.

Referring to FIG. 4a, an electrosurgical device 80 includes an elongated body 82, a pair of arms 84 extending from a distal end of the elongated body 82, and a standard loop electrode 86 connected to the pair of arms 84. The standard loop electrode 86 is a U-shaped wire used for resecting tissue. The standard loop electrode 86 has a distal region 89 and a proximal region 88. The distal region 89 is covered with an insulative coating, and the proximal region 88 is uncovered. In this embodiment, energy is focused at the uncovered proximal region 88 which comes in contact with tissue when performing resection. Alternatively, an upper surface (not shown) of the standard loop electrode 86 may be covered with an insulative coating while the lower surface 87 of the electrode 86 remains uncovered.

Referring to FIG. 4b, an electrosurgical device 70 includes an elongated body 72, a pair of arms 73 extending from a distal end of the elongated body 72, and a horizontal loop electrode 74 connected to the pair of arms 73. The horizontal loop electrode 74 is similar to the standard loop electrode of FIG. 4a except that the horizontal loop electrode 74 is oriented horizontally with respect to the elongated body 72 rather than vertically. The horizontal loop electrode 74 is particularly suitable for use in gynecological applications. The horizontal loop electrode 74 can ablate, cut, and coagulate tissue of endometrial lining. In the embodiment of FIG. 4b, an upper surface 75 of the electrode 70 is coated with an insulative coating while the lower surface 76 remains uncovered to focus energy.

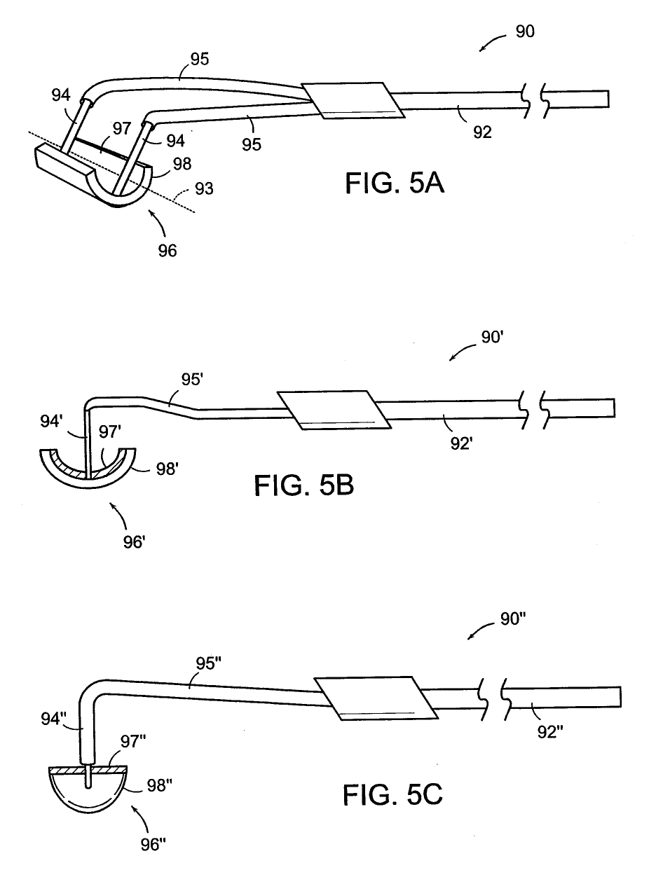

Referring to FIG. 5a, an electrosurgical device 90 includes an elongated body 92, a pair of arms 94 extending from a distal end of the elongated body 92, and a crescent shaped or semi-circular electrode 96 connected to the pair of arms 94. The crescent shaped electrode 96 has a radius of curvature relative to an axis 93 substantially perpendicular to the elongated body 92. The crescent shaped electrode 96 performs substantially the same functions as a roller electrode (i.e., vaporization and coagulation). An advantage of the crescent shaped electrode 96 over a roller electrode is that the amount of conductive surface exposed to an environment such as a saline environment is reduced although the area of the working portion remains the same. The crescent shaped electrode 96, for example, can be formed by attaching half of a metal cylindrical tube to a metal wire (not shown) connected to the pair of arms 94. The electrode 96 has a convex lower surface 98. The upper surface of the crescent shaped electrode 96 may be concave as shown in FIG. 5b or substantially straight as shown in FIG. 5c. The lower surface 98 is the working portion which comes in contact with the tissue during vaporization or coagulation. The area of the lower surface 98 is greater than the area of the upper surface 97. In the embodiment of FIGS. 5b and 5c, the upper surface 97′, 97″, is covered with an insulative coating while the lower surface 98′, 98″ remains uncovered to focus current. In one embodiment, the pair of arms 94 comprise an insulative jacket 95 and the insulative jacket 95 can be extended down to cover as much of the bare wire portion of the pair of arms 94 as possible, as shown in FIG. 5c.

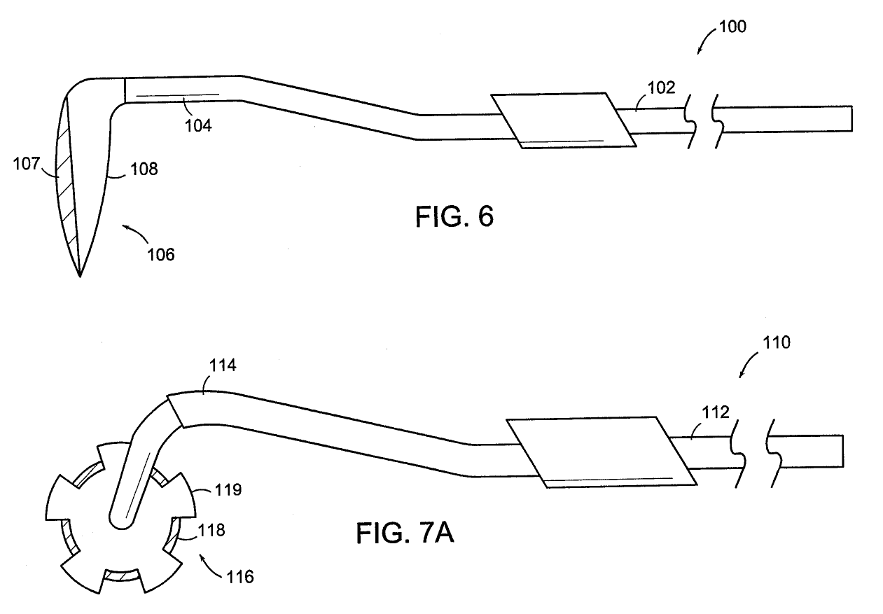

Referring to FIG. 6, an electrosurgical device 100 includes an elongated body 102, a pair of arms 104 extending from a distal end of the elongated body 102, and a knife electrode 106 connected to the pair of arms 104. The knife electrode 106 is used for making incisions in tissue. The knife electrode 106 has a distal region 107 and a proximal region 108. The distal region 107 is covered with the insulative coating, while the proximal region 108 remains uncovered. In this embodiment, energy is focused at the proximal region 108 which comes in contact with tissue when making the incision.

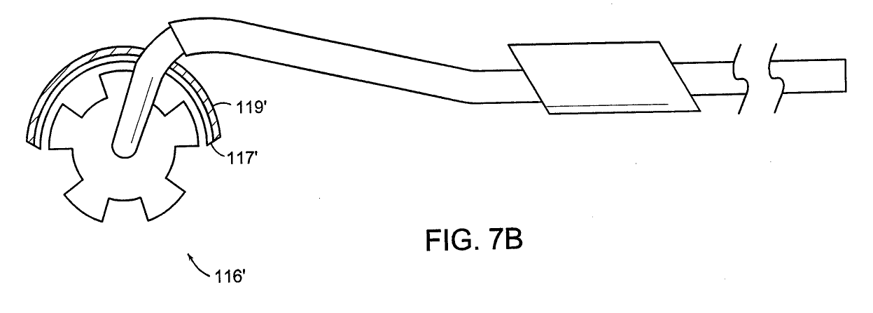

Referring to FIG. 7A, an electrosurgical device 110 includes an elongated body 112, a pair of arms 114 extending from a distal end of the elongated body 112, and a grooved roller electrode 116. The roller electrode 116 has grooves 118 and protrusions 119. The grooves 118 are covered with an insulative coating, while the protrusions 119 remain uncovered. In this embodiment, energy is focused at the protrusions 119, which come in contact with tissue during vaporization.

Referring to FIG. 7B, an electrosurgical device 110′ includes a grooved roller electrode 116′ and a wheel well or a shield 117′ disposed adjacent the electrode 116′, shielding a portion of the electrode surface from the environment. An outer surface of the shield 117′ is coated with an insulator 119′. The electrode 116′ comprises a conductive material.

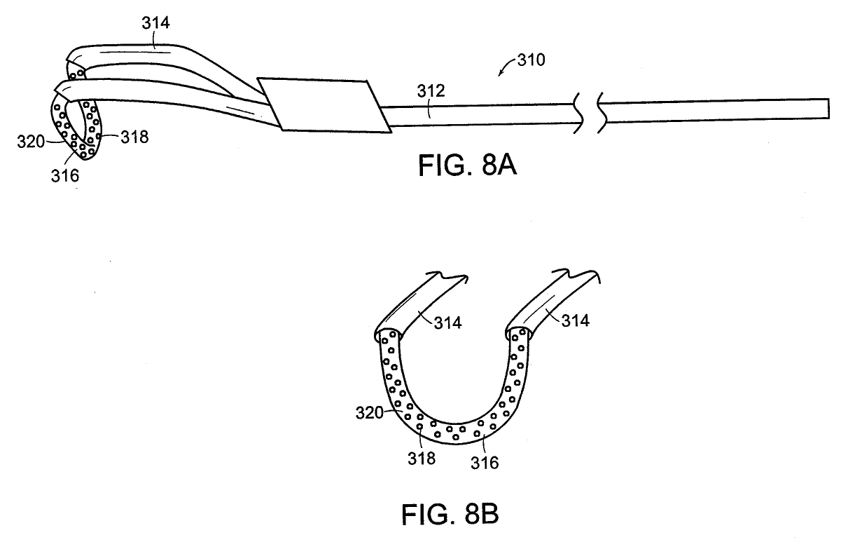

Referring to FIGS. 8a and 8b, an electrosurgical device 310 includes an elongated body 312, a pair of arms 314 extending from a distal end of the elongated body 312, and an electrode 316 in communication with the pair of arms 314. The electrode 316 has a plurality of randomly dispersed conductive regions 318. The conductive regions 318 are created by a non-uniformly deposited insulative coating 320 on the electrode 316. Such non-uniform deposition allows energy emission to preferentially breakthrough the thinner coated regions. In this embodiment, the thickness of the film can be as small as 1 micron, for example and as large as, for example, about 200 microns. It is to be appreciated however, that the thickness of the film in other embodiments can be greater than 300 microns or less than 1 micron. Although the conductive regions 318 are dispersed, the conductive regions 318 are capable of transmitting a current of up to 2 Amps to tissue disposed near the conductive regions 318 in order to perform resection. It is to be appreciated that higher currents can be supplied depending on the intended application.

In another embodiment, the conductive regions 318 can comprise a plurality of pin holes created by the process of vapor deposition of the insulative coating 320 on the electrode, described above. The electrosurgical device can further include a sheath for carrying the elongated body 312 and for delivering an electrolytic non-osmotic fluid such as saline, to a treatment path. In this embodiment, energy applied to the electrode 316 remains focused at the conductive regions 318 when used in conjunction with an electrolytic fluid.

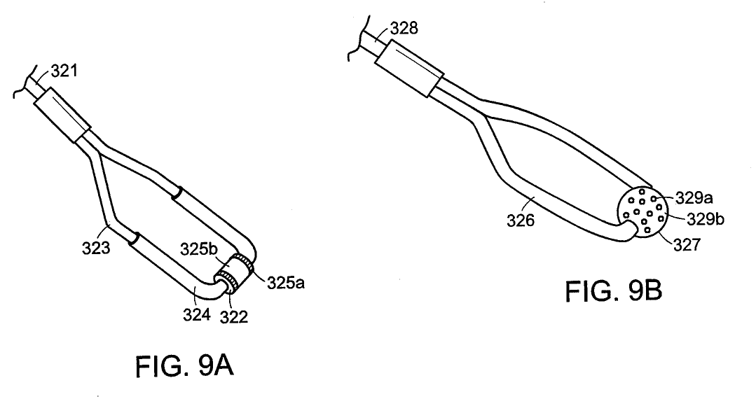

As shown in the embodiment of FIGS. 8a and 8b, the electrode 316 comprises a substantially U-shaped loop electrode. The insulative coating, however, may be placed on other types of electrodes such as a cylindrical roller electrode or a spherical roller electrode, as shown in FIGS. 9a and 9b, respectively.

Referring to the embodiment of FIG. 9a, the electrosurgical device includes an elongated body 321, a pair of arms 323 in communication with the distal end of the elongated body 321, and a cylindrical roller electrode 322 connected to the pair of arms 323. The arms 323 can have an insulative sheath 324 or coating disposed thereon, and the roller electrode 322 can be completely or partially conductive. For example, only the outer portions 325a of the roller electrode 322 can be coated with an insulative coating having a certain resistance to cracking at high temperatures and high voltages. In this regard, energy is focused in the middle of the roller electrode 325b. Alternatively, the roller electrode 327 can include an uneven deposition of insulative coating such as that shown in FIG. 9b.

Referring to the embodiment of FIG. 9b, an electrosurgical device includes an elongated body 328 in communication with a pair of arms 326 at a distal end, and a spherical roller ball electrode 327 connecting the pair of arms 326. The spherical rollerball electrode 327 operates in a similar fashion as described in the embodiment of FIGS. 9a and 9b. The uneven deposition of an insulative coating 329b allows energy to be focused at the conductive regions 329a of the roller ball electrode 327. It is to be appreciated that the embodiments described in FIG. 9a and FIG. 9b can further include a sheath enclosing the elongated body 321, 328 for delivering fluid to the treatment site.

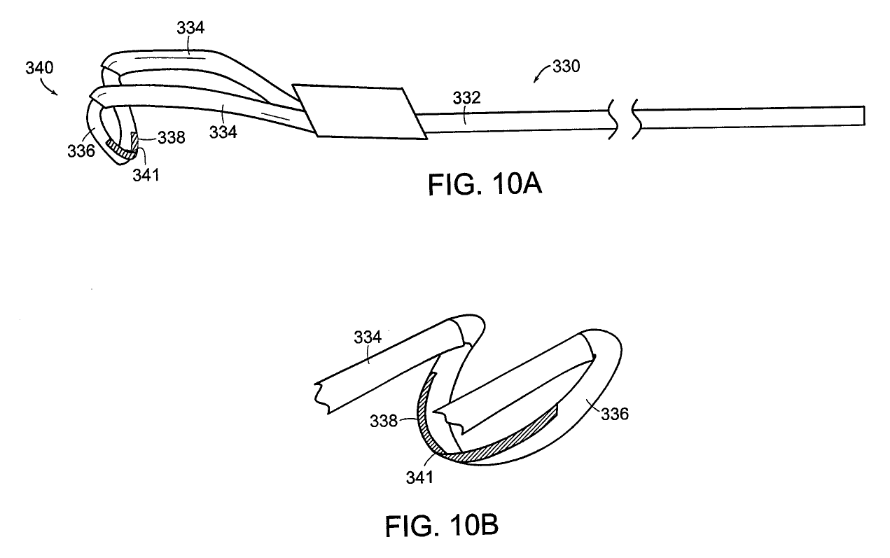

Referring to FIGS. 10a and 10b, an electrosurgical device 330 includes an elongated body 332, a pair of arms 334 extending from a distal end of the elongated body 332, and an electrode 340 in communication with the pair of arms 334. The pair of arms 334 can have an insulative sheath or coating. In this embodiment, the electrode 340 has a first region 336 covered with an insulative coating and a second region 338 covered with graphite. By coating the second region 338 with graphite, the second region 338 is masked while the first region is subsequently coated with the insulative coating. Graphite is placed on the second region 338 by dipping, brushing, and spraying. The graphite covering does not allow the insulator to bond to it, and thus leaves the second region 338 free of insulative coating. The graphite that remains on the second region 338 thereafter disintegrates upon the application of a voltage of greater than 100 volts (peak to peak) at RF frequency to the electrode 340 and exposes a conductive region underneath. Thus the conductive region is exposed and energy is focused at the conductive region during a resection procedure

As shown in the embodiment of FIGS. 10a and 10b, the electrode 340 is a loop electrode having a sharp proximal edge 341 used in resection. The second region 338 comprises an area immediately adjacent the sharp proximal edge 341, and the first region 336 comprises the remainder of the electrode 340. The electrosurgical device 330 can further include a sheath for carrying the elongated body 332 and for delivering a non-osmotic fluid such as saline, glycine or sorbitol to a treatment path. In this embodiment, energy applied to the electrode 340 remains focused at the second region 338 when used in conjunction with a fluid.

In each of the embodiments, the electrosurgical device can be efficiently used with a non-osmotic fluid, such as, for example, saline, glycine or sorbitol. Moreover, the electrosurgical device of the present invention can be used in saline, an electrolytic, non-osmotic fluid without a considerable loss of energy to the tissue undergoing treatment or the fluid. Additionally, the present invention avoids the use of high currents to deliver energy to the treatment site, as energy is effectively focused in the conductive section or sections of the electrode. The result is higher current density, which promotes the generation of a plasma field. In addition, electrodes other than those provided as examples herein can include an insulative coating to expose only a working portion of the electrodes.

In the embodiments of the present invention, a portion of the conductive material of an electrode is covered with an insulative coating to minimize exposure of the conductive member to an environment such as the irrigation fluid, and only a working portion of the electrode is exposed to the environment. The insulative coating disposed on an electrode comprises a material capable of remaining adhered to a conductive material forming the electrode upon application of a voltage of up to about 1000 volts to 2000 volts and upon generation of a plasma field near the electrode. It is to be appreciated that finding the appropriate insulator for the coating is not a trivial matter as most insulators can disintegrate upon generation of plasma fields. A preferred insulator used in the present embodiment has superior electrical resistivity, dielectric strength, and hardness, in addition to having good adhesion to the conductive material forming the electrode. The thickness of the insulative coating covering a non-working portion of an electrode can range from 0.0005 inches to 0.030 inches. An insulative coating that is too thick can create residual stress in the coating, causing the coating to crack and be removed from the electrode. An insulative coating that is too thin may be insufficient to insulate the non-working portion of the electrode. Surface roughness of the insulative coating is less than 50 rms. In a preferred embodiment, the surface roughness is less than 32 root mean square.

In one embodiment, the insulative coating is a diamond-like carbon (DLC) coating sold under the trademark Diamonex® by Diamonex, a unit of Monsanto Company (Allentown, Pa.). DLC is an amorphous diamond material which resembles properties of a naturally occurring diamond. DLC has a hardness in the range from 1000 to 5000 kg/mm², an electrical resistivity in the range from 10⁴ to 10¹² ohms-cm, a dielectric constant of approximately 100 volts (rms) at mains frequency and good adhesion to a substrate.

In one embodiment, DLC is vapor deposited onto the electrode. In other embodiments, the DLC can be deposited by ion beam deposition, RF plasma deposition and by the process of polycrystalline growth. As will be further described, vapor deposition is a microfabrication technology well known to those skilled in the electronics fabrication art. Ion beam deposition technique is described in U.S. Pat. No. 5,508,368, which is incorporated herein by reference. In another embodiment, DLC is deposited using a hot filament chemical vapor deposition technique. The DLC coating is deposited on a working portion of the electrode then removed by etching or other removal processes, such as grinding and EDM (Electrical Discharge Machining) while the DLC coating on the non-working portion remains. In another embodiment, a working portion of the electrode is masked. while DLC is vapor deposited on the electrode, such that DLC coating is prevented from depositing on the working portion of the electrode.

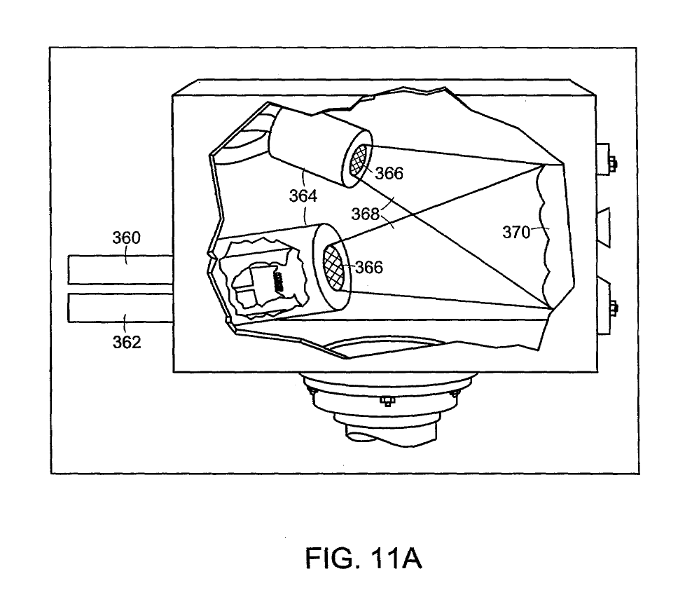

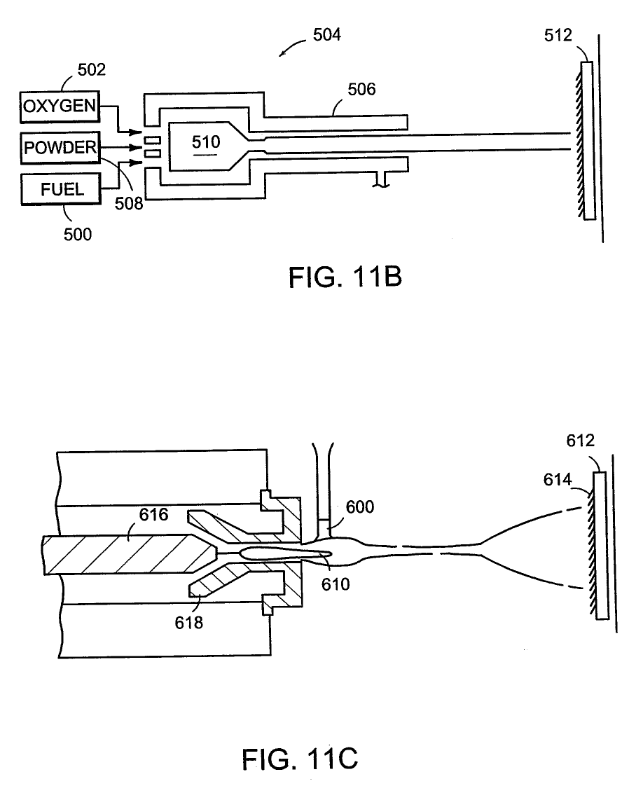

As shown in FIG. 11a, in a dual ion beam deposition process, plasma is generated by applying a mixture of hydrocarbon and argon gases 360, 362 to each ion source 364. Electrically charged grids 366 are placed at one end of the ion source 364. The grids 366 extract and accelerate the hydrocarbon and argon ions 368 toward a substrate 370 to be coated. The substrate 370 is maintained at a temperature between 20° C. and 50° C. as the substrate 370 is sufficiently remote from the plasma within the ion source 364. The accelerated ions 368 combine on the surface of the substrate 370 to produce an amorphous carbon coating. The process causes some of the ions to embed in the substrate 370 thereby providing excellent adhesion. The DLC coating placed on the electrode can have a thickness up to about 10 microns. It is to be appreciated that this thickness can vary depending on the intended application of the device. For example, in one embodiment, the film is evenly deposited and the thickness of the film can vary from about 6 microns to about 10 microns.

In another embodiment, synthetic polycrystalline diamond can be used as insulative coating. Polycrystalline diamond has a thermal conductivity greater than 1000 W/m° K, an electrical resistivity of greater than 10¹¹ ohm-cm, a thermal expansion of about 2×10⁻⁶/° C. between 25° C. and 200° C., a dielectric constant of about 5.7, a dielectric strength of about 300+V/μm, and a shear strength of about 10⁸ N/m².

In still another embodiment, a ceramic coating can be used to cover a non-working portion of the electrode. A ceramic coating is relatively inexpensive, durable, and does not wear over time. Examples of suitable ceramic coatings include alumina, zirconia, and a combination of alumina and titania. The alumina titania combination is particularly useful as the alumina is a good insulator having a high dielectric strength and titania provides toughness to the mixture. The preferred combination includes 87% alumina and 13% titania. The ceramic coating can be placed on the electrode using any one of several methods. In one detailed embodiment, metal disposed over the electrode may be anodized to form a metal oxide layer on the electrode. To grow an alumina layer, for example, aluminum is first disposed on the electrode and the aluminum is allowed to oxidize to form the alumina. The process of anodizing metal to form an oxide is well known to those in the art.

In another detailed embodiment, a ceramic coating is sprayed onto the electrode. Ceramic coating can be sprayed on the electrode using thermal spray. Thermal spraying provides several advantages. Thermal spraying improves adhesion of the ceramic coating to the electrode during use. Ceramic coating placed on a cold electrode tends to crack while the electrode is heated during electrosurgery, due to a difference in the coefficient of expansion between the metal and the ceramic material. Thermal spraying improves adhesion between the electrode and the ceramic coating during use of the electrode, because the ceramic coating is placed over the electrode, while the electrode is expanded under heat. Therefore, when the electrode is heated again during use, the ceramic coating is less likely to crack. Thermal spraying also allows one to control the thickness of the ceramic coating. A coating thickness can range from 0.0005 inches to 0.030 inches.

Examples of suitable thermal spraying techniques include high velocity oxygen fuel (HVOF) spraying, shown in FIG. 11b, and plasma spraying, shown in FIG. 11c. Referring to FIG. 11b, in HVOF spraying, fuel 500 and oxygen 502 are fed into a combustion chamber 504 and combustion is provided. The combustion produces a hot pressure flame 510. The flame is forced down a long nozzle 506 increasing its speed. Powder 508 of a material to be deposited may be fed into the combustion chamber 504 under high pressure or fed through the nozzle 506. The powder within the flame is applied to a substrate 512 to be coated. Referring to FIG. 11c, in plasma spraying, material in the form of powder 600 is injected into a very high temperature plasma flame 610. Plasma flame 610 is generated by passing a gas through a passageway between a cathode 616 and an anode nozzle 618. The material rapidly heats and is accelerated to a high velocity. The material arriving at a substrate 612 to be coated rapidly cools forming a coating 614.

In a preferred embodiment, the electrode is pre-treated to enhance bonding with the ceramic coating and the ceramic coating is thermal sprayed onto the electrode. The electrode can be pre-treated in a number of ways. In one detailed embodiment, the electrode surface is roughened to create better mechanical bonding between the electrode and the coating. For example, the electrode surface can be sand blasted to create the rough surface. Alternatively, metal can be sprayed onto the electrode surface to create the rough surface. In another detailed embodiment, a bond coating is sprayed on the electrode prior to coating the electrode. The bonding coating, for example, may comprise nickel chromium super alloy.

In still another embodiment, the electrodes described in FIGS. 1-10B may be formed of a ceramic material, and a metal layer is deposited on the ceramic electrode form the conductive working region. The metal layer can be first deposited and then etched to form the conductive working region. Metal deposition and etching techniques are well known to those skilled in the art.

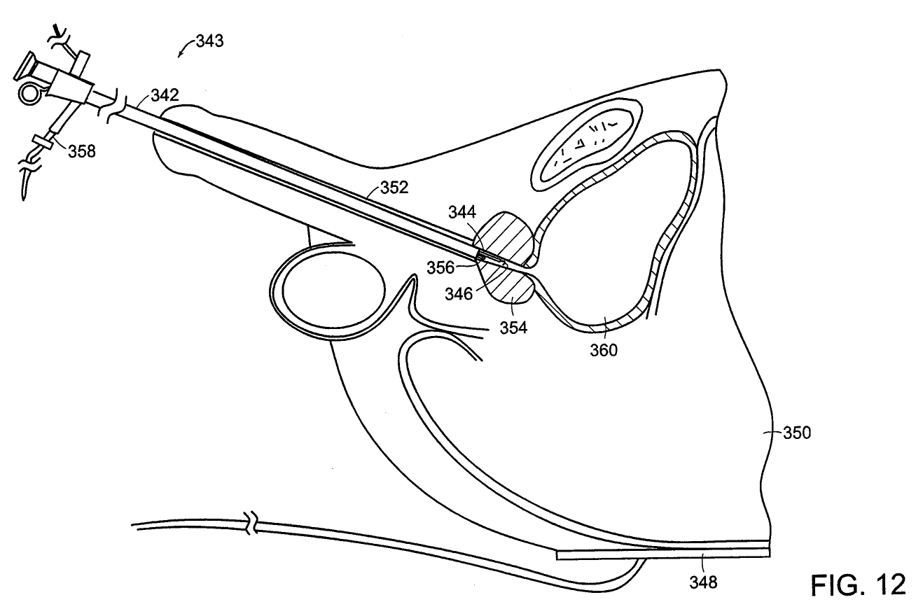

Referring to FIG. 12, a resectoscope assembly 343 includes a resectoscope 342 defining a channel (not shown) and an electrosurgical device 344 insertable through the channel. The electrosurgical device 344 may be of any embodiment described above with reference to FIGS. 1-10B. As illustrated in FIG. 12, in a typical transurethral procedure, a return electrode 348 is positioned on a surface of the body 350 and the resectoscope assembly 342 is inserted inside the urethra 352. The electrosurgical device 344 is inserted through the channel of the resectoscope 342 and positioned along a treatment path near prostate tissue 354 to be resected. The resectoscope 342 includes a telescope 356 at a distal end, such that the electrosurgical device 344 can be positioned under observation. The tissue to be treated is flushed with a non-osmotic fluid introduced through a luer port 358 for injecting fluid. In a preferred embodiment, the non-osmotic fluid can be a non-osmotic, electrolytic fluid such as saline. Alternatively, the non-osmotic fluid can be a non-osmotic, non-electrolytic fluid such as glycine or sorbitol. A voltage in the range from about 1000 volts to 2000 volts (peak to peak) is applied across the working electrode 346 and the return electrode 348 to generate a plasma field, without embedding the working electrode 346 inside the prostate tissue 354 when resecting the tissue. The working electrode 346 is moved along the treatment path to resect and coagulate the prostate tissue 354.

Although a resection procedure using the resecting electrode of the present invention has been described with reference to FIG. 12, resection of tissues other than prostate tissues can be performed according to the invention. For example, the resectoscope assembly 343 can be inserted deeper into the bladder 360 to resect bladder tissues. Alternatively, the resectoscope assembly 343 can be inserted inside a female patient to resect or ablate a tumor from the walls of the uterus or to resect an endometrium lining. In addition, bipolar electrodes in addition to monopolar electrodes can be selectively coated with an insulative coating for limiting current distribution according to the invention.

It is to be appreciated that the use of an insulative coating such as a DLC coating or a ceramic coating can have other applications. For example, biopsy forceps can be selectively coated with an insulative coating to prevent the biopsy sample from being damaged. The inner surfaces of the biopsy forcep that comes in contact with the removed biopsy sample can be coated with the insulative coating, while the outer surfaces of the forceps used to remove the sample can remain conductive.

Those skilled in the art will be able to make numerous uses and modifications of and departures from the embodiments described herein without departing from the invention. Consequently, other embodiments are within the following claims.

What is claimed is:

1-21. (canceled)

22. An electro-surgical device, comprising:
an elongated body adapted to be coupled to a source of energy at a proximal end;
a pair of arms extending from a distal end of the elongated body; and
an electrode in communication with the pair of arms, the electrode having a plurality of conductive regions crated by an insulative coating disposed on the electrode.

23. The electro-surgical device of claim 22, wherein the plurality of conductive regions are randomly dispersed on the electrode.

24. The electro-surgical device of claim 22, wherein the insulative coating is non-uniformly deposited on the electrode.

25. The electro-surgical device of claim 22, wherein the insulative coating comprises a diamondlike carbon coating.

26. The electro-surgical device of claim 22, wherein the insulative coating is vapor deposited onto the electrode according to one of the following procedures: vapor deposition, ion deposition, RF deposition, or polycrystalline growth.

27. The electro-surgical device of claim 22, wherein the conductive regions are capable of transmitting a current of up to 2 Amps to tissue disposed adjacent the conductive regions.

28. The electro-surgical device of claim 22, wherein the electrode comprises a substantially U-shaped loop.

29. The electro-surgical device of claim 22, wherein the electrode comprises a cylindrical roller electrode.

30. The electro-surgical device of claim 22, wherein the resecting electrode comprises a spherical roller electrode.

31. The electro-surgical device of claim 22, further comprising a sheath for carrying the elongated body and for delivering a non-osmotic fluid to a treatment path.

32. The electro-surgical device of claim 31, wherein the non-osmotic fluid comprises saline.

33. The electro-surgical device of claim 31, wherein the non-osmotic fluid comprises glycine.

34. An electro-surgical device, comprising:
an elongated body adapted to be coupled to a source of energy at a proximal end;
a pair of arms extending from a distal end of the elongated body; and
an electrode in communication with the pair of arms, the electrode having a plurality of randomly dispersed conductive regions created by a non-uniformly deposited insulative coating thereon.
